(19) United States
(12) Patent Application Publication
(10) Pub. No.: US 2020/0275835 A1
(43) Pub. Date: Sep. 3, 2020

(54) SYSTEM AND METHOD FOR CONTACT LESS CONTROL OF AN APPLIANCE TECHNICAL FIELD

(71) Applicant: SANDEEP KUMAR CHINTALA, SURREY (GB)

(72) Inventors: Sandeep Kumar CHINTALA, SURREY (GB); Sriranhan RASAKATLA, HYDERABAD (IN)

(21) Appl. No.: 16/646,817

(22) PCT Filed: Sep. 7, 2018
(86) PCT No.: PCT/GB2018/052536

§ 371 Date: Mar. 12, 2020

(30) Foreign Application Priority Data
Sep. 13, 2017 (GB) 201741032515

(51) Int. Cl. A61B 5/00; A61B 5/0476; A61B 5/04; A61B 5/11

(52) U.S. Cl. CPC A61B 5/0006; A61B 5/0024; A61B 5/0476; A61B 5/04012; A61B 2562/0219; A61B 5/6822; A61B 5/6814; A61B 5/6824; A61B 5/11

(57) ABSTRACT

A wearable device is provided for contactless operation of remotely controlled appliance present inside an environment. The wearable device may include a one motion sensor to generate a motion signal based on a sensed movement of a body part of the user bearing the wearable device. In addition, the wearable device includes a brainwave sensor to generate an EEG signal indicative of brain activity of the user. Further, the wearable device includes a processor that may select an appliance from amongst the plurality of remotely controllable appliances based on the motion signal and the EEG signal and control the selected remotely controlled appliance based on the EEG signal over a wireless network.

### TECHNICAL FIELD

The subject matter described herein, in general, relates to human-machine interaction technologies, and in particular, but not exclusively, to a non-contact gesture-based control technique for controlling an electronic device.

### BACKGROUND

Technologies for contact-less interaction between humans and machines aim at providing control and operation of an electronic or electrical appliance without physical contact between a user and the appliance. Such technologies include gesture-based control, voice-based control, electroencephalogram (EEG) signal based control, or the like. For instance, the gesture-based technology works on a principle of sensing a gesture made by the user and operating the appliance in response to the gesture. On the other hand, EEG-based control may involve sensing brain or nerve signals, referred to as EEG signals, corresponding to the user's brain activity and controlling the appliance based on the sensed signals.

### BRIEF DESCRIPTION OF DRAWINGS

The above and other features, aspects, and advantages of the subject matter will be better understood with regard to the following description, appended claims, and accompanying drawings where:

FIG. 1 illustrates an environment having remotely controlled appliances, in accordance with one implementation of the present subject matter.

FIG. 2a different designs of a wearable device, in accordance with one implementation of the present subject matter.

FIG. 2b illustrates the wearable device, in accordance with one implementation of the present subject matter.

FIG. 3 illustrates a method for contactless operation of the remotely controlled appliances, in accordance with one implementation of the present subject matter.

FIG. 4 illustrates a network environment, in accordance with one implementation of the present subject matter.

### DETAILED DESCRIPTION

Conventional techniques use one type of the signal to operate the appliance. For example, gesture based control techniques utilizes motion signal to select and operate the appliance. In another example, EEG based control techniques utilizes EEG signals to select and operate the appliance. However, conventional techniques do not verify the selection based on the input signal. As a result, the selection made using conventional techniques are not accurate. Moreover, conventional techniques do not use information related to a position the user with respect to the appliance. There may be case where when two appliances are operated based on a single gesture. Since the conventional techniques did not determine if the user is in the vicinity of the first appliance or the second appliance, this may lead to a conflict so to which appliance the user wants to operate when the user makes the gesture to control one of the appliances. Thus, conventional contacts less controllers are less accurate in their operation.

The present subject matter relates to various aspects for providing contact less control of the appliances with precise selection. Techniques based on the present subject matter utilize two different types of signals to select an appliance. For example, motion of a user's body part is sensed and motion signals generated corresponding to the sensed motion, is used to select the appliance. In addition, the brain activity related to the same body part is also sensed and an EEG signal corresponding to the sensed movement is also used to select the appliance. Moreover, selections made using the motion based signal and the EEG signal are cross verified to finalise the selection of the appliance. Since the present subject matter utilizes different sources to select the appliance, the selection of the appliance is accurate.

One aspect of the present subject matter employs brainwave sensors that may sense the user's brain activity. In one example, the brainwave sensor may generate a first EEG signal based on a movement of a user's first body part, such as the user's arm, and the first EEG signal may facilitate in determining an orientation of the first body part so as to determine an appliance to which user intend to select. In addition, the brainwave sensor may generate a second EEG signal based on a movement of user's second body part, such as movement of fingers, and the second EEG signal may facilitate in determining a predefined control action for the appliance towards which the user is pointing. The present subject may also employ motion sensors that sense motion of the user's body part onto which the wearable device is worn. In one example, selection of the appliance based on the motion signal is performed in parallel to the selection made using the first EEG signal Since the selection made by the signal EEG signal is complemented by the selection made by the motion signal, the selection of the appliance made based on the present subject matter is accurate.

Another aspect of the subject matter relates to measuring a strength of a wireless signal transmitted by the appliance. Further, the strength of the wireless signal is indicative of a distance between the user and the appliance. Thus, a measure of the strength of the wireless signal is used to determine a distance between the user and the appliance. Thus, the techniques based on the present subject matter can determine the position of the user with respect to the appliance and accordingly may operate the appliance when the user is around the appliance.

The manner in which the wearable device operates shall be explained in details with respect to the accompanying figures. It should be noted that the description merely illustrates the principles of the present subject matter. It will thus be appreciated that those skilled in the art will be able to devise various arrangements that, although not explicitly described herein, embody the principles of the present subject matter and are included within its spirit and scope. Furthermore, all examples recited herein are principally intended expressly to be only for pedagogical purposes to aid the reader in understanding the principles of the present subject matter and the concepts contributed by the inventor(s) to furthering the art, and are to be construed as being without limitation to such specifically recited examples and conditions. Moreover, all statements herein reciting principles, aspects, and embodiments of the present subject matter, as well as specific examples thereof, are intended to encompass equivalents thereof.

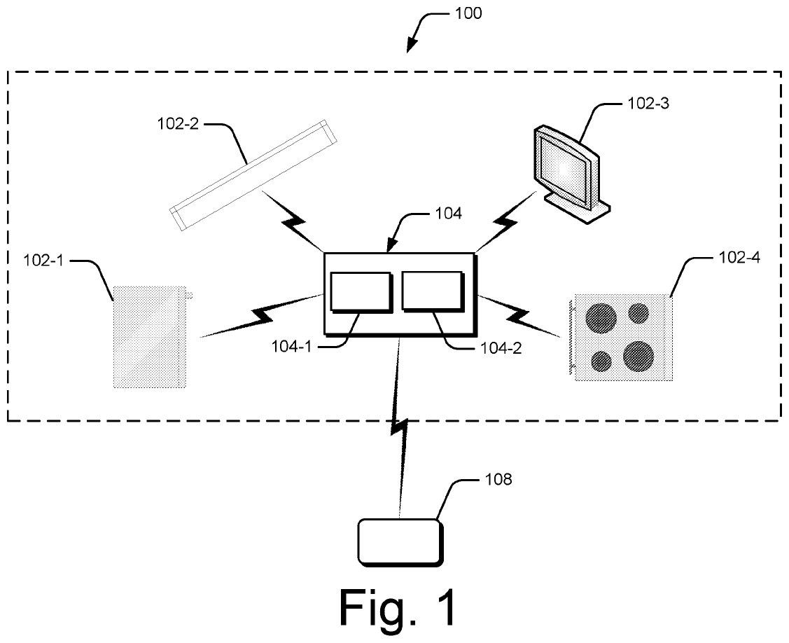

FIG. 1 illustrates an environment 100, in accordance with one implementation of the present subject matter. The environment 100 is a space where a user can exist and can operate the components present in the environment 100. In one example, the environment 100 can be a room in a house, an office area, or the like. Further, it may be understood that different environments may exist. The environment 100 may include multiple remotely-controlled appliances 102-1, 102-2, 102-3; collectively referred to as 102 hereinafter. The remotely-controlled appliances 102 can be, but not limited to, a television, air conditioner, lighting devices, or the like. Generally, each of the remotely controlled appliances may include an appliance controller 104 for controlling an operation of the remotely controlled appliance 102. In another implementation, the environment 100 may include a single appliance controller 104 that may be connected to all the remotely controlled appliances 102. The appliance controller can be a handheld computing device, such as a smartphone, tablet, PDA, or the like. According to an aspect, the appliance controller 104 may include a microcontroller 104-1 that may perform control operation on the remotely controlled appliance 102. In addition, the appliance controller 104 may also include a wireless module 104-2 that may receive instructions to control the remotely controlled appliance 102 via a wireless network connection. In one example, the wireless module 104-1 can be a Bluetooth transceiver or a Wi-Fi module. In the illustrated aspect, the appliance controller 104 may include other components, such as a power source to provide power the appliance controller 104, switches for manual operation of the remotely controlled appliance 102, and relay switches to control power surges in the remotely controlled appliance 102.

According to an aspect, the environment 100 may also include a wearable device 108 that may be borne and used by a user to control various remotely controlled appliances 102 present in the environment 100. In one example, the wearable device 108 may control the remotely controlled appliances 102 over the wireless network established between the wearable device 108 and the appliance controller 104. For example, in case the first remotely controlled appliance 102-1 is a television, the wearable device 108 may turn ON/OFF, increase/decrease the volume, change the channel, or the like on the first remotely controlled appliance 102-1 based on the user's input. In the illustrated aspect, the wearable device 108 may select the remotely appliance 102 based on different types of inputs or signals. In one example, the wearable device 102 may use brainwave signals from the user's brain activity as well as gestures made by the user's body part to select the remotely controlled appliance 102. Further, use different signals for selecting the remotely controlled appliance 102 at the same time makes the selection of the remotely controlled appliance 102 accurate. In addition, the wearable device 108 may also determine presence of the wearable device 100 in the environment 100. Further, detection the presence of the wearable device 108 in the environment and the remotely controlled appliances 102 facilitates in improving the accuracy of the operation of the wearable 108. The constructional details of the wearable device 108 and the manner by which the wearable device 108 operates will be explained later.

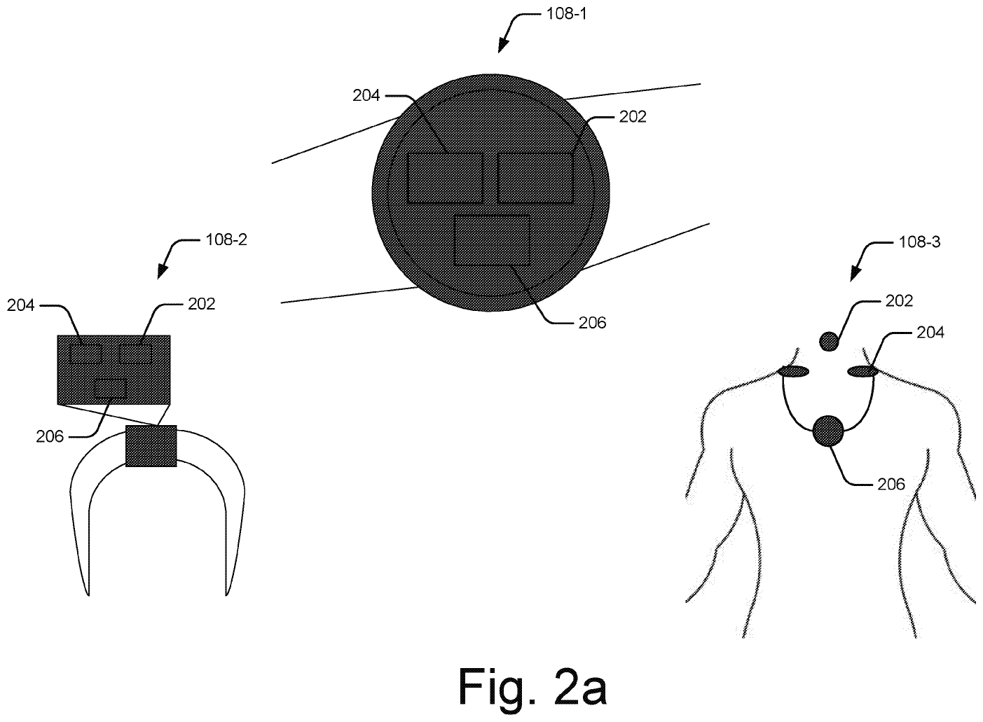

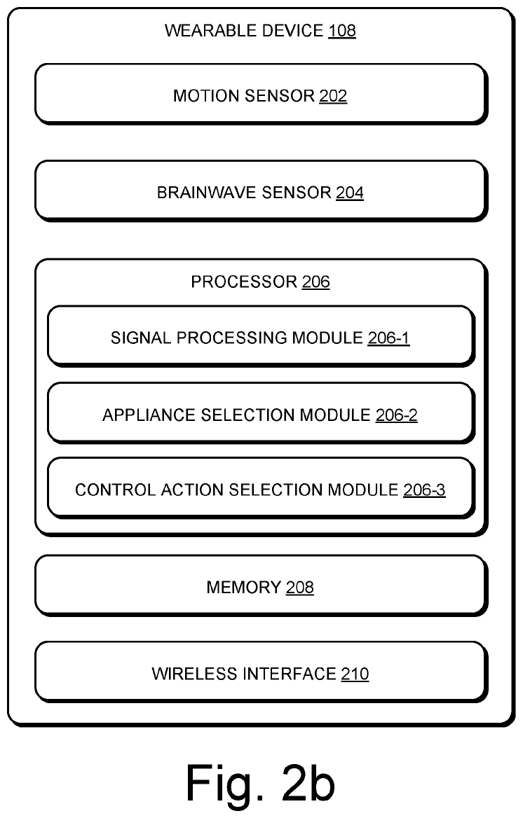

FIGS. 2a and 2b illustrates a wearable device 108, in accordance with one implementation of the present subject matter. The wearable device 108 can be used to control the remotely controlled appliances 102 based on user's brain activity and body gestures. In an example, the wearable device 108 can be borne by the user for contactless interaction with a plurality of remotely controlled appliances present in the environment 100. For instance, the wearable device 108 can be formed as a wrist-worn device 108-1, a headgear 108-2, or a necklace 108-3. In the illustrated aspect, the wearable device 108 may include a motion sensor 202 that can sense a motion of the user's body part. For example, the motion sensor 202 in the wrist borne wearable device 108-1 may sense a gesture made by an arm of the user. Accordingly, the motion sensor 202 may generate a motion signal indicative of the sensed movement of the user's body part on which the wearable device 108 is worn. In case of the head borne wearable device 108-2, the motion sensor 202 may sense a movement of head. As may be understood, the wearable device 108 may include multiple motion sensors 202 to sense motion of the user. In one example, the motion sensor 202 can be an inertial measurement sensor (IMU), accelerometer, gyroscope, or the like.

According to an aspect, the wearable device 108 may include a brainwave sensor 204 that may sense activities of the user's brain. For example, the brainwave sensor 204 may sense brain activities corresponding to the gesture made the user's body part. In addition, the brainwave sensor may sense the brain activities related to the control action for the remotely controlled appliances 102. Accordingly, the brainwave sensor 204 may generate an electroencephalogram (EEG) signal. In an example, the brainwave sensor 204 can include EEG pads installed inside the wrist borne wearable device 108-1 may pick brain signals travelling through the nerves of the user. In another example, the EEG pads inside the head borne wearable device 108-2 may make contact with the scalp of the user and may pick brain signals from the scalp. In yet another example, the EEG pads in the neck borne wearable device 108-3 may be installed to be in the proximity of the nerves when worn around the neck. The brainwave sensor 204 may pick a variety of signals from the nerves of the user, including the signal that relates to a control action for the appliance.

The wearable device 108 may also include a processor 206 that may be operably coupled to the motion sensor 202 and the brainwave sensor 204. The processor 206, in operation, may process the signals received from the motion sensor 202 and the brainwave sensor 204 to select the remotely controlled appliance and determine the control action for the selected remotely controlled appliance 102. The processor 206 can be a single processing unit or a number of units, all of which could include multiple computing units. The processor 206 may be implemented as one or more microprocessors, microcomputers, microcontrollers, digital signal processors, central processing units, state machines, logic circuitries, and/or any devices that manipulate signals, based on operational instructions. In the illustrated implementation, the processor 206 may include a signal processing module 206-1. The signal processing module 206-1, in operation, may process the inputs signals coming from the motion sensor 202 and the brainwave sensor 204 for further processing. In one example, the signal processing module 206-1 may separate the EEG signals corresponding to the gesture made by the user's body part and an EEG signal corresponding to the control action from the gamut of EEG signals received from the brainwave sensor 204. In another example, the signal processing module 206-1 may also measure a strength of a wireless signal transmitted by the wireless module 104-2. Further, the signal processing module 206-1 may uses the measured strength of the wireless signal to determine if the wearable device 108 is present in the environment 100. The manner by which the signal processing module 206-1 operated will be discussed later.

According to an aspect, the processor 206 may include an appliance selection module 206-2 that may select the remotely controlled appliance 102 based on the signals processed by the signal processing module 206-1. In one example, the appliance selection module 206-1 may process the motion signal to determine the remotely controlled appliance 102. In addition, the appliance selection module 206-2 may process the EEG signal corresponding to the gesture of the user's body part to select the remotely controlled appliance. The appliance selection module 206-2 may further cross verify the selection made using the motion sensor and the EEG signal to finalise the selection. The processor 206 may also include control action selection module 206-3 that may determine the control action based on the EEG signal corresponding to the control action. The manner by which the appliance selection module 206-2 and the control action selection module 206-3 operate in conjunction with the signal processing module 206-1 would be explained in details later.

In one example, the processor 206 may further be coupled to a memory 208 which may further include computer readable instructions for the processor 206, reference information and signals related to spatial orientation of the remotely controlled appliance 102 in the environment 100. In addition, the memory 208 may store predefined signals related to control actions associated with all the remotely controlled appliances 102 in the environment 100. Although the memory 208 is shown as a separate component from the processor 206, the memory 208 can be in-built inside the processor 206. In operation, the data stored in the memory 208 is accessed by the processor 206 for selecting the appliance and thereafter controlling the appliance based on the user's input. In another example, the processor 206 may append or update the data present in the memory 208 to recognize the gesture made by the user and the control action for the remotely controlled appliances 102. Further, updating the data present in the memory 208 may be a part of training of the wearable device 108 to recognize and record the gesture. The manner by which the memory 208 is updated will be discussed later.

According to an aspect, the wearable device 108 may also include a wireless interface 210 that may communicate with the appliance over a wireless network. The wireless interface 210 can be a Wi-Fi or a Bluetooth transmitter, or both. In one example, the wireless interface 210 may perform two operations. First, the wireless interface 210 may establish a wireless network connection with the RF module 104-1 of the appliance. In one example, the wireless connection can be a Bluetooth link or Zigbee link or Wi-Fi link. Second, the wireless module 108 may relay the control instructions to the appliance controller 104.

According to an aspect, the wireless module 108 may also sense a strength of the wireless signal from the remotely controlled appliance 102. Strength of the signal may be used to determine the position of the wearable device 108. Determining the position of the wearable device 108 with respect to the appliance may facilitate in determining if the wearable device 108 is present in the environment 100. Further, presence of the wearable device 108 in the environment 100 may facilitate the wearable device 108 in the remotely controlled appliances 102 present in the environment 100 from other remotely controlled appliance present in the other environments. The recognition may further prevent in conflict in the operation of the remotely controlled appliance 102 in case any other remotely controlled appliance 102 that can be operated by the same gesture.

In the illustrated example, the wearable device 108 constantly monitors a strength of the wireless signal transmitted by the appliance controller 104. Further the strength of the received signal is proportional to the distance between a transmitter and a receiver. In one example, the wearable device 108 maps the signal strength with a library of reverse signal strength indicators (RSSI) to determine if the wearable device 108 is present in the environment 100. The RSSI may be understood as is a measure of the strength of the in a received signal. For example, the strength of the signal sensed by the wearable device 108 will be strong when the wearable device 108 is present in the environment 100. In case, the wearable device 108 is not present in the environment 100 or the user wearing the wearable device 108 travels from the environment 100 to another environment (not shown), the strength of the signal from the appliance controller 104 in the environment 100 would get weak. Simultaneously, strength of the signals from a appliance controller present in the other environment would grow strong. Thus change in signal strength facilitates to the determine the position of the wearable device 108. Accordingly, the wearable device 108 can be configured to operate the remotely controlled appliances present in the other environment.

For the following description explaining the operation of the wearable device 108, the wearable device 108 is borne by the user on his/her wrist and the wearable device 108 is present in the environment 100. Prior to the operation, the memory 208 is loaded with reference signal corresponding to the spatial orientation of all the remotely controlled appliances 102. In addition, the memory 208 is loaded with a library of predefined signals corresponding to all possible control actions for all the remotely controlled appliance 102. The memory 208 is also loaded with the RSSI.

In order to select the remotely controlled appliance 102, the user may raise and point the arm toward the appliance 102 to be selected. As the user swings the arm, the motion sensor 202 senses the movement of the arm and accordingly, generates a motion signal. The motion signal is then communicated to the processor 206.

Simultaneously, the brainwave sensor 204 senses the nerve signals travelling through the user's nerve corresponding to the movement of the arm and generate the EEG signals. In one example, the signal from the brain includes the signals for the arm's muscle to raise the arm. In addition, the nerve signal includes signals for individual fingers of the user's hand. In the illustrated implementation, the brainwave sensor 204 may sense different nerve signals and accordingly, generates different EEG signals. As mentioned previously, the brainwave sensor 204 may generate a first EEG signal based on the movement of the user's first body part. The brainwave sensor 204 may also generate a second signal based on a movement of a second part of the user. For example, the user may swing his/her arm that corresponds to the selection of the remotely controller appliance 102 and also moves the fingers to make a gesture that corresponds to the control action. In such a scenario, the brainwave sensor 204 generates the first EEG signal based on the movement of the arm and generates the second EEG signal based on the movement of the fingers. In addition to the first and the second EEG signal, the brainwave sensor 204 also generates additional EEG signals based on the. Once generated, all the EEG signals are relayed to the processor 206 for further analysis.

Once the motion signal and the EEG signals are received, the processor 206 may determine the remotely controlled appliance 102 to be selected based on the motion signal. In one example, the signal processing module 206-1 may receive the motion signals and EEG signals and process the signal for further analysis. For example, the signal processing module 206-1 may remove attenuation present in the both the signals. Once the signals are processed, the signal processing module 206-1 proceeds to selected the remotely controlled appliance 102.

In an example, the signal processing module 206-1 process the EEG signals to select the remotely controlled appliance 102. In an example, the processor 206 may distinguish the first signal and the second signal from a gamut of EEG signals received from the brainwave sensor 204. In one example, the processor 206 may perform independent component analysis (ICA) on the EEG signals. The ICA can be understood as a statistical technique for decomposing complex dataset, which in this case, are EEG signals into individual signals. Generally, ICA may include steps, for example, centering the dataset, performing singular vector decomposition of the dataset to identify the Eigen values and other matrices. As mentioned previously, the EEG signal received by the processor 206 includes various signals including the first EEG signal and the second EEG signal. In one example, the processor 206 may separate the first EEG signal and the second EEG signal from the mixture of EEG signals.

Once the signal processing module 206-1 distinguishes between the first EEG signal and the second EEG signal, the appliance selection module 206-2 processes the distinguished signals separately to select the remotely controlled appliance 102. In one example, the processor 206 may search for a reference signal stored in the memory 210 corresponding to the first EEG signal. Once a match is found, the processor 206 determines the remotely controlled appliance 102 to be controlled. In addition, the processor 206 also maps the motion signal with a reference signal that corresponds to the spatial orientation of the remotely controlled appliance 102. For example, the motion signal is mapped to the reference signal which corresponds to the x, y coordinates of the position of the remotely controlled appliance 102. Thereafter, the appliance selection module 206-2 verifies if the remotely controlled appliance 102 determined based on the first EEG signal is the same as the remotely controlled appliance 102 determined based on the motion signal.

Once verified, the control action selection module 206-3 determines the control action for the selected remotely controlled appliance 102. In the illustrated implementation, the control action selection module 206-3 search through the database in the memory 208 to find a predefined signal that corresponds to the second EEG signal. For example, the second EEG signal may relate to a pinch gesture made by the user's finger. Accordingly, the control action selection module 206-3 would search for the predefined signal corresponding to the pinch gesture made for the selected remotely controlled appliance 102. Once a match is found, the second EEG signal is mapped to the predefined signal. Based on the mapping, the control action selection module 206-3 determines the control action for the selected remotely controller appliance 102. In one example, the control action associated with the selected remotely controlled appliance 102 is to switch ON the selected remotely controlled appliance 102.

Once the selection of the remotely controlled appliance 102 and determination of the control action is complete, the processor 206 communicates relevant information to the wireless interface 210. In an example, the relevant information includes the instructions to establish communication with the selected remotely controlled appliance 102 and the control action to be performed on the selected remotely controlled appliance 102. Once the relevant information is received by the wireless interface 210, the wireless interface 210 establishes a wireless connection with the selected remotely controlled appliance 102. In an example, the wireless interface 210 may communicate with the RF module 104-1 (shown in FIG. 1) of the appliance controller 102 to establish a wireless connection. As mentioned previously, the communication can be established based on any wireless protocol known in the art. Once the communication is established, the wireless interface 210 relays the control actions to the RF module 104-1. Upon receiving the control actions, the RF module 104-1 communicates the control action to the microcontroller 104-1. The microcontroller 104-1 then operates the remotely controlled appliance based on the control action.

In an additional aspect of the present subject matter, the wearable device 108 may also determine the position of the wearable device 108 with respect to the remotely controlled appliance 102 in the environment 100. There may be a case where the user may move between different environment 100. In such scenario, the wearable device 108 can determine the position with respect to the environment 100. In one example, the wearable device 108 may measure a strength of the signal transmitted by the appliance controller 104. As may be understood, the strength of the signal received by the wearable device 108 is sensed by the wireless interface 210 and the strength is communicated to the processor 208. As the wearable device 108 moves away from the environment 100, the strength sensed by the wearable device 108 weakens. Simultaneously, the processor 208 constantly map the sensed strength against library of RSSI stored inside the wireless interface 210. Once the processor 208 finds a match of the reference signal with an RSSI indicative of the exit of the wearable device 108 from the environment 100, the processor 206 determines that the wearable device 108 has moved out of the environment 100.

Simultaneously, the processor 208 may also map a strength of a signal coming from the second appliance controller of the second environment and checks if the strength of the signal is growing strong as the wearable device 108 moves between the environments. Hereto, the wearable device 108 maps the received signal with the RSSI to check if the wearable device 108 has entered into the second environment 100. Once the processor 208 finds a match of the reference signal with an RSSI indicative of the entry of the wearable device 108 from the environment 100, the processor 206 determines that the wearable device 108 has moved into the second environment 100.

According to one implementation of the present subject matter, the wearable device 108 can also be trained to record and recognize gestures related to the aforementioned operation. In one example, the wearable device 108 store information regarding the gesture performed by the user during training of the wearable device 108 in the memory 208. During the training operation, the user performs the gesture which is to be recorded. As the user performs the operation, the brainwave sensor 204 senses the brain activity and accordingly, generates the EEG signals. The EEG signals are then communicated to the processor 206. Thereafter, the processor 206 performs the ICA to distinguish the first EEG signal and the second EEG signal from the rest of the EEG signals. Once distinguished, the processor 206 records the first EEG signal and the second EEG signals in the memory 208 as a reference signal and a predefined signal respectively for the gesture performed by the user. In one example, the processor 206 records the first EEG signal and the second EEG signal as a time varying function of the electrical signal stored in the memory 208.

Once recorded, same operation is conducted to obtain a concurrent reading. For example, the recording operation is performed 5-6 times in order to obtain a concurrent reading. Once a concurrent reading is obtained, the concurrent reading is stored as the reference signal for the gesture.

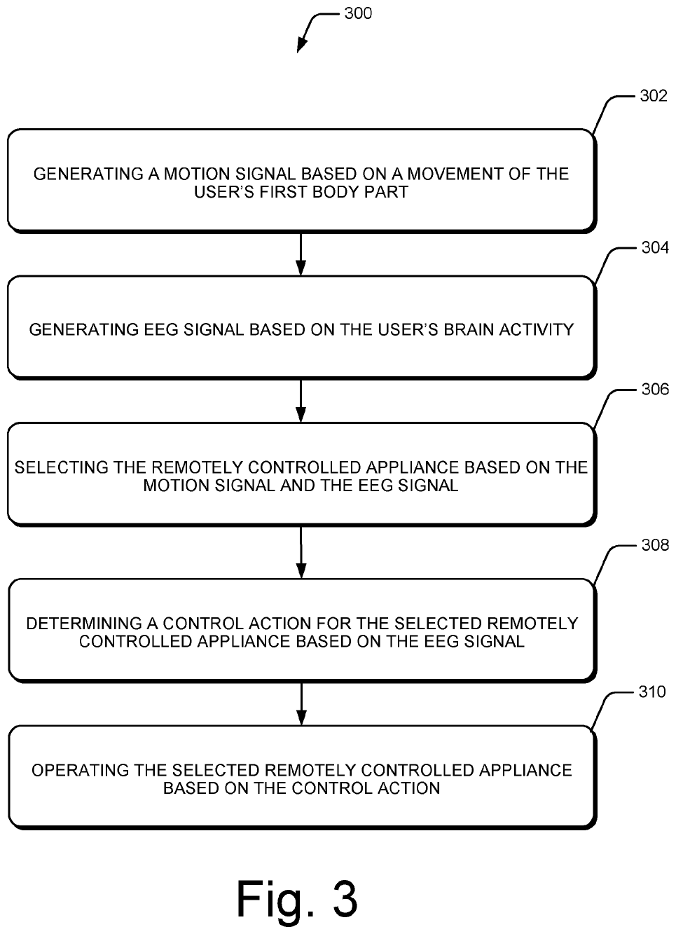

FIG. 3 illustrates a method 300 for contactless interaction with a plurality of remotely controlled appliances 102 present in an environment 100 by a wearable device 108 worn by a user. Any number of the described method blocks can be combined in any order to implement the method 300, or any alternative methods. Additionally, individual blocks may be deleted from the method 300 without departing from the scope of the subject matter described herein. The method 300 is described with reference to the wearable device 108 of FIGS. 1, 2a, and 2b.

At block 302, a motion signal corresponding to the user's first body part is generated by the motion sensor 202. For example, the motion sensor 208 senses when the user swings the arm and/or points the finger towards the remotely controlled appliance 102. Accordingly, the motion sensor generates the motion signal. The motion signal is then communicated to the processor 206.

At block 304, EEG signals related to the brain activity of the user is measured by the brainwave sensor 204. As mentioned previously, the brainwave sensor 204 may generate a first EEG signal based on the movement of the user's first body part. The brainwave sensor 204 may also generate a second signal distinct based on a movement of a second body part of the user. Accordingly, the brainwave sensor 204 generates the EEG signals and communicates the EEG signals to the processor 206.

At block 306, the remotely controlled appliance to be controlled, is selected. The selection is performed by using the motion signal and the EEG signal. In one example, the processor 206 may map the motion signal with a reference signal that corresponds to the spatial orientation of the remotely controlled appliance 102. For example, the motion signal is mapped to the reference signal which corresponds to the spatial orientation of the remotely controlled appliance 102 in the environment 100.

In addition, the processor 206 processes the EEG signals to select the remotely controlled appliance 102. In an example, the processor 206 may distinguish the first signal and the second signal from a gamut of EEG signals received from the brainwave sensor 204. As described previously with respect to FIGS. 2a and 2b, the processor 206 may separate the first EEG signal and the second EEG signal. Once distinguished, the processor 206 may search for a reference signal in the memory 210 corresponding to the first EEG signal. Once a match is found, the processor 206 determines the remotely controlled appliance 102 to be controlled. Thereafter, the processor 206 check if the remotely controlled appliance 102 determined based on the first EEG signal is the same as the remotely controlled appliance 102 determined based on the motion signal.

At block 308, a control action for the selected remotely controlled appliance 102 is determined. In one example, the second EEG signal is mapped against a predefined signal which corresponds to the motion of the user's second body part.

At block 310, the selected remotely controlled appliance 102 is operated based on the determined controlled action. In one example, the processor 206 communicates information to the wireless interface 210 and accordingly, the wireless interface 210 establishes a wireless connection with the RF module 104-1 to communicate the control action. Once the control action is communicated, the appliance controller 104 operates the remotely controlled appliance 102.

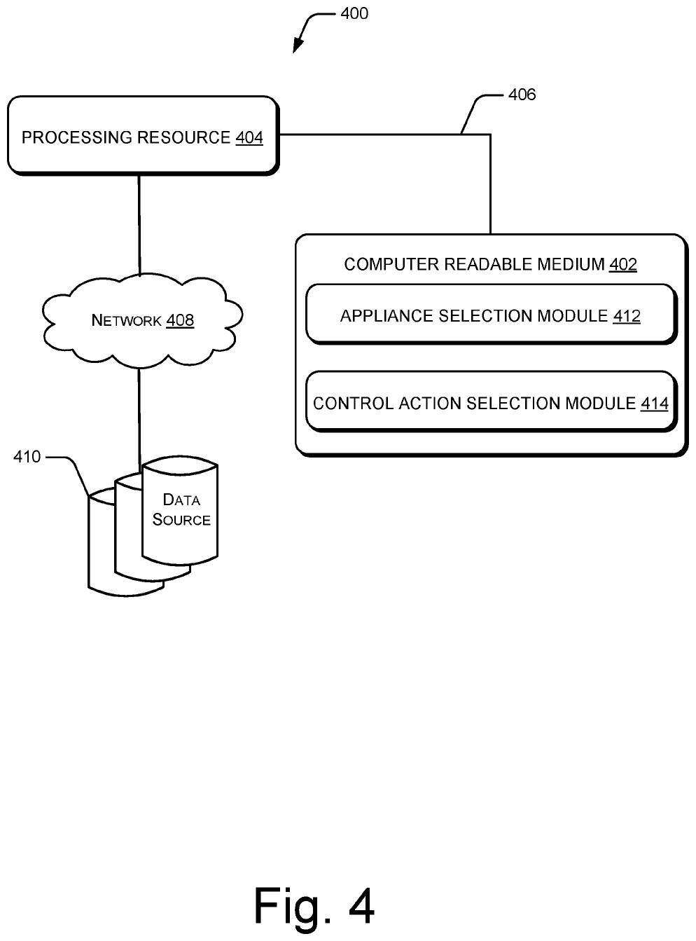

FIG. 4 illustrates a network environment 400 using a non-transitory computer readable medium 402 for contactless operation of the remotely controlled appliances 102 (shown in FIG. 1), in accordance with one implementation of the present subject matter. In one example, the network environment 400 includes a processing resource 404 communicatively coupled to the non-transitory computer readable medium 402 through a communication link 406.

For example, the non-transitory computer readable medium 402 can be, for example, an internal memory device or an external memory device. In one example, the communication link 406 may be a direct communication link, such as one formed through a memory read/write interface. In another example, the communication link 406 may be an indirect communication link, such as one formed through a network interface. In such a case, the processing resource 404 can access the non-transitory computer readable medium 402 through a network 408. The network 408 may be a single network or a combination of multiple networks and may use a variety of communication protocols.

The processing resource 404 and the non-transitory computer readable medium 402 may also be communicatively coupled to data source 410. The data source 410 can include, for example, databases and computing devices. The data source 410 may be used by the database administrators and other users to communicate with the processing resource 404.

In one example, the non-transitory computer readable medium 402 includes a set of computer readable instructions, such as appliance selection module 412 and a control action selection module 414. The set of computer readable instructions, referred to as instructions hereinafter, can be accessed by the processing resource 404 through the communication link 406 and subsequently executed to perform acts for contactless operation.

For discussion purposes, the execution of the instructions by the processing resource 404 is similar to the processor 206 that has been described with reference to various components introduced earlier with reference to description of FIGS. 1, 2a and 2b.

On execution by the processing resource 404, the appliance selection module 412 maps the motion signal and the EEG signal against the reference signal stored in the memory 208 to determine the remotely controlled appliance 102. In one example, the appliance selection module 412 maps the received motion signal against the reference signal that corresponds to the spatial orientation of the remotely controlled appliance 102.

In addition, the control action selection module 414 analyzes the EEG signal to determine the control action for the selected remotely controlled appliance 102. In one example, the control action selection module 414 maps the EEG signal against predefined signals stored in the data source 410 to determine the control action corresponding to the EEG signal.

Once the remotely controlled appliance 102 is selected and the control action is determined, the communication module 416 communicates the relevant information to the wireless interface 210 to operate the selected remotely controlled appliance 102. The communication module 416 can also determine the position of the remotely controlled appliance 102. The communication module 416 maps a strength of the signal of the RF module 104, received by the wireless interface 210, against the RSSI to determine if the wearable device 108 is present the environment 100 (shown in FIG. 1).

Although the present subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter.

1. A wearable device to be borne by a user for contactless interaction with a plurality of remotely controlled appliances present in an environment, the wearable device comprising:
at least one EEG brainwave sensor operative to:
generate a first EEG signal based on detection of a movement of a first body part of the user bearing the wearable device;
generate a second EEG signal based on detection of a movement of a second body part of the user; and
a processor operably coupled to the at least one EEG sensor, and the plurality of remotely controllable appliances, wherein the processor is operative to:
select a remotely controlled appliance from amongst the plurality of remotely controllable appliances based on the first EEG signal; and
control the selected appliance based on the second EEG signal.

2. The wearable device as claimed in claim 8, wherein the processor includes a memory that stores a spatial orientation of each of the remotely controlled appliance present in the environment.

3. The wearable device as claimed in claim 9, wherein the processor determines position of the remotely controlled appliance with respect to the wearable device based on the spatial orientation of the remotely controlled appliance.

4. The wearable device as claimed in claim 1, wherein the device is wearable on one of a wrist of the user, around a neck of the user, and on a head of the user.

5. A wearable device to be borne by a user for contactless interaction with a plurality of remotely controlled appliances present in an environment, the wearable device comprising:
at least one motion sensor operative to generate a motion signal based on a sensed movement of a body part of the user bearing the wearable device;
at least one brainwave sensor operative to generate an electroencephalogram signal indicative of brain activity of the user;
a processor operably coupled to the at least one motion sensor, the at least one EEG sensor, and the plurality of remotely controllable appliances, wherein the processor is operative to:
select a remotely controlled appliance from amongst the plurality of remotely controllable appliances based on the motion signal and the EEG signal; and
control the selected remotely controlled appliance based on the EEG signal over a wireless network.

6. The wearable device as claimed in claim 5, wherein the processor includes a memory that stores a spatial orientation of each of the remotely controlled appliance present in the environment.

7. The wearable device as claimed in claim 6, wherein the processor determines position of the remotely controlled appliance with respect to the wearable device based on the spatial orientation of the remotely controlled appliance in the environment.

8. The wearable device as claimed in claim 5, wherein the motion sensor is an inertial measurement unit.

9. The wearable device as claimed in claim 5, wherein the wearable device is wearable on one of a wrist of the user, around a neck of the user, and on a head of the user.

10. A method for contactless interaction with a plurality of remotely controlled appliances present in an environment by a wearable device worn by a user, the method comprising:
sensing, by at least one motion sensor inside the wearable device, a gesture made by a body's part on which the wearable device is worn and generating a corresponding motion signal;
sensing, by at least one brainwave sensor inside the wearable device, a brain activity of the user and generating a corresponding EEG signal
selecting, by a processor inside the wearable device and coupled to at least one motion sensor and at least one EEG sensor, the remotely controlled appliance to be operated based on the sensed gesture and the EEG signal, wherein selection comprises mapping the motion signal and the EEG signal with a reference signal corresponding to a spatial orientation associated with the remotely controlled appliance;
determining, by the processor, a control action for the selected appliance based on the EEG signal, wherein determining comprising mapping the EEG signal with a predefined signal associated with the control action; and
operating the selected appliance by the wearable device based on the determined control action.

11. The method as claimed in claim 10, wherein selecting comprises:
measuring, by the processor, strength of a wireless signal transmitted by the remotely controlled appliance;
mapping the of the wireless signal with a reference signal associated with spatial orientation of the remotely controlled appliance; and
determining a position of the wearable device with respect to the remotely controlled appliance based on the mapping.

12. A non-transitory computer-readable medium comprising instructions executable by a processor to:
receive a motion signal from at least one motion sensor, wherein the motion signal corresponds to sensed movement of a body part of a user by the motion sensor;
receive an EEG signal from at least one brainwave sensor, wherein the EEG signal corresponds to brain activity of the user;
select a remotely controlled appliance, from amongst a plurality of remotely controlled appliance present in an environment based on the received motion signal; and
determine a control action for the selected remotely controlled appliance based on the received EEG signal.

13. The non-transitory computer-readable medium as claimed in 12 further comprising instructions executable by the processor to:
measure strength of a wireless signal transmitted by the remotely controlled appliance;
map the of the wireless signal with a reference signal associated with spatial orientation of the remotely controlled appliance; and
determine a position of the wearable device with respect to the remotely controlled appliance based on the mapping.
